(19) United States
(12) Patent Application Publication
(10) Pub. No.: US 2026/0363784 A1
(43) Pub. Date: Nov. 19, 2026

(54) SYSTEMS AND METHODS FOR TIME TO TARGET ESTIMATION FROM IMAGE CHARACTERISTICS

(71) Applicant: KONINKLIJKE PHILIPS N.V., EINDHOVEN (NL)

(72) Inventors: Leili SALEHI, WALTHAM, MA (US); Ayushi SINHA, CLEVELAND HEIGHTS, OH (US); Javad FOTOUHI, CAMBRIDGE, MA (US); Sean Joseph KYNE, BROOKLINE, MA (US); Ramon Quido ERKAMP, SWAMPSCOTT, MA (US); Vipul Shrihari PAI RAIKAR, SOMERVILLE, MA (US)

(21) Appl. No.: 19/121,850

(22) PCT Filed: Oct. 18, 2023
(86) PCT No.: PCT/EP2023/078907

§ 371 Date: Apr. 17, 2025

Related U.S. Application Data
(60) Provisional application No. 63/418,750, filed on Oct. 24, 2022.

(51) Int. Cl. A61B 34/20; A61B 34/00; A61B 34/10; A61B 34/30; A61B 34/37; A61B 90/00

(52) U.S. Cl. CPC A61B 34/20; A61B 34/10; A61B 34/25; A61B 34/37; A61B 90/39; A61B 2034/107; A61B 2034/2065; A61B 2034/252; A61B 2034/303; A61B 2090/3966

(57) ABSTRACT

A system for navigating an interventional instrument in an anatomy of a patient. The system receives one or more images including a portion of the anatomy of the patient and an interventional instrument disposed within the portion of the anatomy. The system identifies, from the one or more images, a location of the interventional instrument in the portion of the anatomy and an anatomical feature of the portion of the anatomy. The system further identifies a target region of interest (ROI) in the anatomy. The system predicts a time to navigate the interventional instrument from the location of the interventional instrument, through the portion of the anatomy, to the location of the target ROI based on the anatomical feature of the portion of the anatomy.

### FIELD

The following relates generally to the endovascular arts, device tracking arts, artificial intelligence (AI) arts, and related arts.

### BACKGROUND

Ischemic stroke is an emergency medical condition, in which a clot or other vascular blockage is preventing blood flow to brain tissue. A common treatment is an intravascular procedure in which a catheter or other an intravascular instrument is inserted, and its tip moved to the location of the vascular blockage and used to remove the blockage. This process of removing the blockage by an intravascular procedure is sometimes referred to as revascularization since it effectively restores vascular blood supply to the tissue. While an ischemic stroke is one example of such an emergency situation, similar revascularization can be appropriate therapy for treating blockages in other critical anatomy (heart, lungs, et cetera). Revascularization delay in ischemic stroke or other emergency blockage situations can result in long term and sometimes irreversible disabilities or even life-threatening consequences. Navigation from access point to the target area is not always straightforward and may need a lot of focus on specific regions of the navigation path, which can take a long time. Monitoring a total and remaining navigation time in emergency procedures such as ischemic stroke revascularization may better inform decisions like changing devices or navigation strategies, and is critical for a successful treatment. For non-emergency procedures, having information about the navigation time to target could help the staff in preparing the room for the upcoming steps including readying the right personnel and equipment for the next procedure step.

Reducing a delay in treating emergency neurovascular diseases such as ischemic stroke significantly increases the chance of positive procedure outcomes and shortens the rehabilitation period (see, e.g., Tapuwa D. Musuka MBChB, Stephen B. Wilton M D, Mouhieddin Traboulsi M D, Michael D. Hill M D, “Diagnosis and management of acute ischemic stroke: speed is critical. ,” CMAJ, Sep. 8, 2015, 187(12)).

One of the steps towards revascularization for stroke treatment which impacts the total procedure time is intravascular navigation of the tip of the interventional instrument to the target vessel or region of interest (ROI). Typically, this is done under the guidance of a real-time interventional imaging modality such as X-ray or ultrasound imaging. Usually, the interventional imaging provides two-dimensional (2D) images since it can be difficult or impossible to obtain three-dimensional (3D) tomographic images in real-time or with the physician positioned closely to the patient to operate the interventional instrument. However, sometimes multi-view imaging may be used, which can provide some 3D image information. In some cases, a pre-operative 3D image may be available, such as a computed tomography (CT) angiography image, but in an emergency situation there may not be time to obtain such a 3D pre-operative image. Navigation time can significantly increase when the patient has either a tortuous anatomy or multiple bifurcations in the navigation path. The tortuosity of the vasculatures could also lead to multiple device exchanges which could increase the risk of injury in addition to increase in procedure time. Hence, it is important for physicians to maintain precision during endovascular navigation, and this is facilitated by zooming in on the displayed real-time interventional image around the distal end of their device in order to see details related to geometrical complexities (e.g., bends, bifurcations) as they navigate.

However, focusing on a small region of interest (ROI) around the distal end of the device may result in losing the big picture of the vasculature and the remaining path to the target. Consequently, this can also result in losing track of the time required to reach that target which also facilitates decisions during the procedure (e.g., change of staff, modifying the navigation speed, changing devices, etc.). Viewing a small ROI also increases the likelihood that the physician may take a wrong path at a vascular bifurcation.

The following discloses certain improvements to overcome these problems and others.

### SUMMARY

In some embodiments disclosed herein, a system for navigating an interventional instrument in an anatomy of a patient includes a processor and memory. The processor is configured to: receive one or more images including a portion of an anatomy of a patient and an interventional instrument disposed within the portion of the anatomy; identify, from the one or more images, a location of the interventional instrument within the portion of the anatomy and at least one anatomical feature of the portion of the anatomy; identify a target region of interest (ROI) in the anatomy; and predict a time to navigate the interventional instrument from the location of the interventional instrument, through the portion of the anatomy, to a location of the target ROI based on the at least one anatomical feature of the portion of the anatomy.

In some embodiments disclosed herein, a method for navigating a interventional instrument in an anatomy of a patient includes: receiving one or more images including a portion of an anatomy of a patient and an interventional instrument disposed within the portion of the anatomy; identifying, from the one or more images, a location of the interventional instrument within the portion of the anatomy and at least one anatomical feature of the portion of the anatomy; identifying a target ROI in the anatomy; and predicting a time to navigate the interventional instrument from the location of the interventional instrument, through the portion of the anatomy, to a location of the target ROI based on the at least one anatomical feature of the portion of the anatomy.

In some embodiments disclosed herein, anon-transitory computer-readable storage medium has stored a computer program comprising instructions. The instructions, when executed by a processor, cause the processor to: receive one or more images including a portion of an anatomy of a patient and an interventional instrument disposed within the portion of the anatomy; identify, from the one or more images, a location of the interventional instrument within the portion of the anatomy and at least one anatomical feature of the portion of the anatomy; identify a target region of interest (ROI) in the anatomy; and predict a time to navigate the interventional instrument from the location of the interventional instrument, through the portion of the anatomy, to a location of the target ROI based on the at least one anatomical feature of the portion of the anatomy.

In some of these embodiments, the at least one anatomical feature of the portion of the anatomy includes at least one of vessel tortuosity, a bifurcation, a vessel bend, small vessel diameter, and vessel branching in the portion of the anatomy. In some of these embodiments, a trained machine-learning model is applied to predict the time to navigate from the location of the interventional instrument, through the portion of the anatomy, to the location of the target ROI based on the at least one anatomical feature of the portion of the anatomy. In some of these embodiments, a tip of the interventional instrument comprises a radiopaque material and the processor is further configured to identify the location of the interventional instrument in the one or more of images based on the radiopaque tip. In some of these embodiments, a path is determined to navigate the interventional device from the location of the interventional instrument, through the portion of the anatomy, to the location of the target ROI; and a plurality of successive ROIs is identified along the path and predict a time to navigate to each of the plurality of successive ROIs.

One advantage resides in reducing delays during endovascular procedures.

Another advantage resides in monitoring a tip of an endovascular device during an endovascular procedure.

Another advantage resides in using deep learning to monitor a tip of the endovascular device during an endovascular procedure.

Another advantage resides in using imaging to monitor a tip of the endovascular device during an endovascular procedure.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an endovascular device in accordance with the present disclosure.

FIG. 2 diagrammatically illustrates a method of performing a vascular therapy method using the device of FIG. 1.

FIG. 3 diagrammatically shows operation of a machine-learning model receiving as input images with a known location of the tip of an interventional instrument and an identified location of a target, and outputs an estimated time to target.

FIG. 4 diagrammatically shows a visualization displayed by the device of FIG. 1.

### DETAILED DESCRIPTION

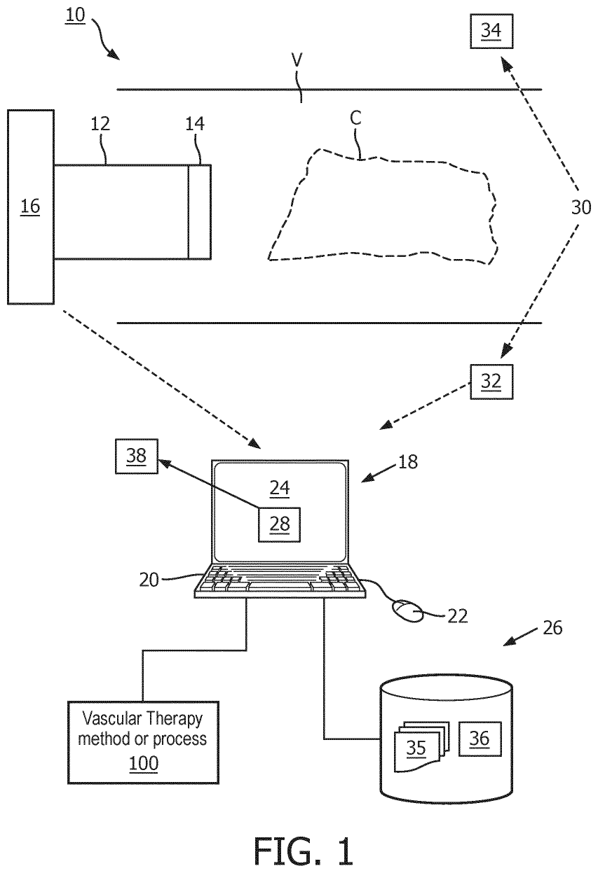

With reference to FIG. 1, a system 10 is diagrammatically shown. The system 10 can be, for example, an endovascular system, an endobronchial system, a surgical system, or any other medical system. As shown in FIG. 1, the system 10 includes an interventional instrument 12 (e.g., a catheter, a probe, a needle, an electrode, and so forth-diagrammatically shown in FIG. 1 as a line) configured for insertion into a portion of anatomy of a patient. For example, the interventional instrument may be configured for insertion into a patient's blood vessel V which has an occlusion or clot C (diagrammatically shown in FIG. 1 with dashed lines). In some embodiments, the interventional instrument 12 is radiopaque. The interventional instrument 12 includes a tip 14. In some embodiments, the tip 14 is radiopaque to improve imaging of the tip in fluoroscopic imaging. The radiopaque tip 14 may be comprised of a different material from the rest of the interventional instrument 12. For example, the tip 14 may comprise a short radiopaque wire (e.g., a platinum or Nitinol wire) that is metallurgically bonded (e.g., by welding) to the end of the interventional instrument 12. Alternatively, in some embodiments, radiopaque markers (not shown), such as platinum tungsten-filled polyurethane bands may be disposed at intervals along the tip 14. In yet another embodiment, the interventional instrument 12 may be constructed to be entirely radiopaque. For example, a wire may be disposed the length of the interventional device and coated with a radiopaque coating such as a tantalum coating.

In some embodiments, a clinician may control movement of the interventional instrument 12 in the blood vessel V. Other embodiments provide robotic control of the movement of the interventional instrument 12. FIG. 1 also shows an embodiment with a robot 16 (diagrammatically shown in FIG. 1 as a box) operatively connected to the interventional instrument 12. The robot 16 may be configured to control movement of the interventional instrument 12 into, through, and out of the blood vessel V. In some embodiments, a clinician may control the robot using controllers, such a joystick or mouse clicks on a user interface, and, in other embodiments, the an autonomous control system may be provided that is configured to automatically steer the robot 16.

FIG. 1 further shows an embodiment in which a processing device 18, such as a computer, controls the robot 16 to automatically move the interventional instrument 12 through the vessel V. The processing device 18 may also include a server computer or a plurality of server computers, e.g., interconnected to form a server cluster, cloud computing resource, or so forth, to perform more complex computational tasks. The processing device 18 may include components, such as a processor 20 (e.g., a microprocessor or other hardware processor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 may be a separate component from the processing device 18. In some embodiment, the processing device 18 may include two or more display devices.

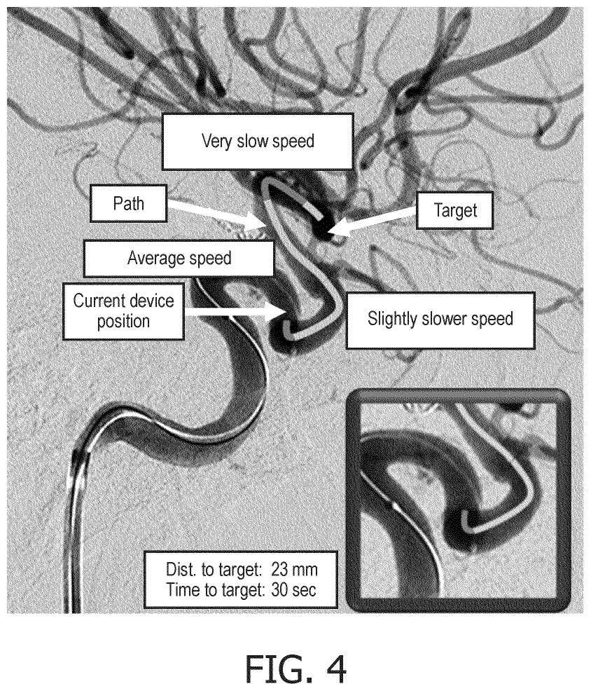

The processor 20 is operatively connected with one or more non-transitory storage media 26. The non-transitory storage media 26 may, by way of non-limiting illustrative example, include one or more of a magnetic disk, RAID, or other magnetic storage medium; a solid-state drive, flash drive, erasable read-only memory (EEROM) or other memory; an optical disk or other optical storage; various combinations thereof; or so forth; and may be for example a network storage, an internal hard drive of the electronic processing device 18, various combinations thereof, or so forth. It is to be understood that any reference to a non-transitory medium or media 26 herein is to be broadly construed as encompassing a single medium or multiple media of the same or different types. Likewise, the processor 20 may be embodied as a single processor or as two or more processors. The non-transitory storage media 26 stores instructions executable by the processor 20. The instructions may include instructions to generate a visualization of a graphical user interface (GUI) 28 for display on the display device 24 (see, e.g., FIG. 4).

FIG. 1 also shows an imaging device 30 configured to acquire a time sequence of images or imaging frames 35 of the movement of the interventional instrument 12 (including the tip 14 of the interventional instrument 12). The interventional instrument may also be referred to herein as an interventional device. In the illustrative example of FIG. 1, the imaging device 30 is a fluoroscopic imaging device (e.g., an X-ray imaging device, C-arm imaging device, a CT scanner, or so forth) and the radiopaque tip 14 of interventional instrument 12 is visible under the fluoroscopic imaging. The imaging device 30 of FIG. 1 is configured to perform real-time imaging. For example, the imaging device 30 may acquire images at a frame rate of 15-60 frames/second (i.e., 15-60 fps) in some nonlimiting illustrative embodiments.

The imaging device 30 in FIG. 1 is in communication with the processor 20 of the electronic processing device 18. As shown in FIG. 1, the imaging device 30 comprises an X-ray imaging device including an X-ray source 32 and an X-ray detector 34, such as a C-arm imaging device. In other embodiments, the imaging device may comprise another modality, such as ultrasound (US), computed tomography (CT), magnetic resonance imaging (MRI), nuclear imaging, etc. In embodiments, the field of view of the imaging device 30 is large enough to encompass at least the tip 14 of the interventional instrument 12 and the vasculature leading to the occlusion or clot C. For embodiments with non-fluoroscopic imaging devices, the radiopaque tip 12 may be replaced by a tip of a type that is observable in the chosen imaging modality. For example, if ultrasound is used for monitoring, then the radiopaque tip 12 may be made of a material, or includes markers of a material, that provides good contrast in ultrasound images. The images 35 captured by the imaging device 30 may be stored in the non-transitory storage media 26.

The processor 20 of FIG. 1 is configured, as described above, to perform process 100, which may be a vascular diagnosis method, vascular therapy method, an endobronchial diagnosis method, an endobronchial therapy method, or a surgical method. The non-transitory storage medium 26 stores instructions which are readable and executable by the processor 20 to perform disclosed operations (or process 100) including performing, for example, a vascular therapy method. In some examples, the method may be performed at least in part by cloud processing.

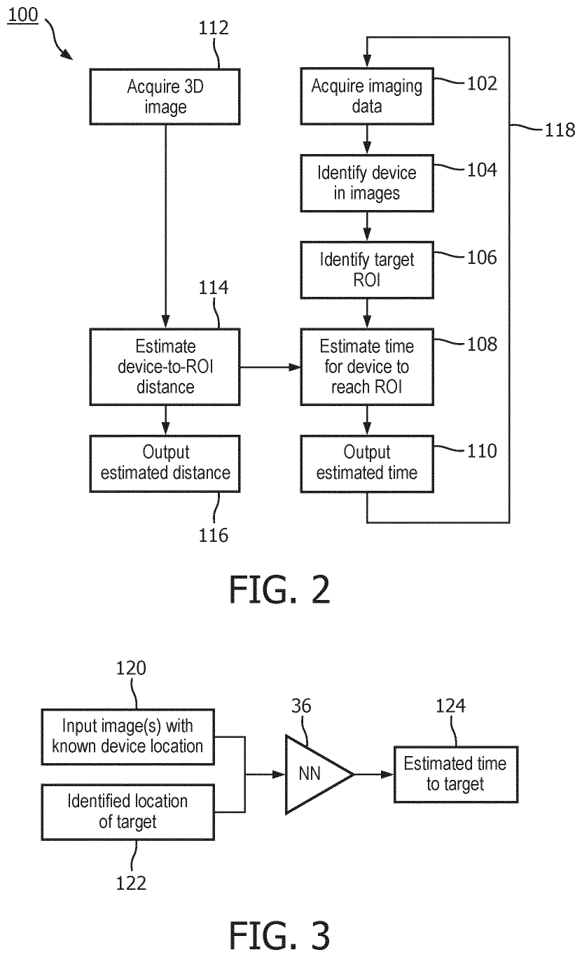

Referring to FIG. 2, and with continuing reference to FIG. 1, an example method 100 is diagrammatically shown as a flowchart. To begin the method 100, the interventional instrument 12 is inserted into the blood vessel V by a clinician and/or using the robot 16.

At operation 102 of method 100, the imaging device 30 performs interventional imaging during an interventional procedure to acquire a time sequence of images 35 (or imaging frames) of the movement of the interventional instrument 12 (with tip 14) during the procedure. In some embodiments, this interventional imaging 102 is performed during the interventional procedure to provide visual guidance to the physician or clinician manipulating the interventional instrument 12 or robot 16 to move the interventional instrument 12 toward a target region of interest (ROI). In some embodiments, the target ROI is an occlusion or clot C in the vessel V. The time sequence of images 35 include the interventional instrument 12 (and tip 14 of the interventional instrument 12) and a portion of the anatomy in which the interventional instrument 12 is disposed (e.g., a vessel V or portion of vasculature leading to the target ROI). In some embodiments, the field of view of the images encompass both the location of the tip 14 and the target ROI, while in other cases the field of view encompasses the location of the tip 14 and a portion of the vasculature between the current location of the tip 14 and the target ROI, but does not encompass the target ROI itself (at least until the tip 14 is navigated sufficiently closer to the region of interest). The time sequence of images 35 is transferred to the processing device 18.

At operation 104 of method 100, processor 20 identifies the location of the interventional instrument 12 in the portion of the anatomy in the time sequence of images 35 (e.g., in a vessel V included in the time sequence of images 35). In some embodiments, processor 20 may identify the location of the interventional instrument 12 by identifying the location of the tip 14 (e.g., radiopaque tip) of the interventional instrument in the portion of the anatomy in the time sequence of images 35.

At operation 106 of method 100, processor 20 identifies the location of the target ROI in the anatomy relative to the location of the interventional instrument 12 (e.g., the tip 14). In some embodiments, the processor 20 may generate a path from the location of the tip 14 to the location of the target ROI. It will be appreciated that the order of the operations 104 and 106 can be swapped, or if sufficient computing capacity is available then the operations 104 and 106 can be performed concurrently. The processor may also identify features of the anatomy that is between the location of the interventional instrument 12 and the location of the target ROI.

At operation 108 of method 100, processor 20 predicts the traversal time for the interventional instrument 12 to traverse (pass through) the portion of the anatomy (e.g., vessel V) to reach the target ROI (e.g., the tip 14 reach the target ROI). In some embodiments, the processor 20 may determine a path between the location of the interventional instrument 12 and the location of the target ROI. In some embodiments, the processor 20 may predict the traversal time by estimating a distance of the interventional instrument 12 (e.g., tip 14) relative to the target ROI across a series of imaging frames in the time sequence of images 35. In some embodiments, the processor 20 may predict the traversal time by estimating a velocity of the interventional instrument 12 (e.g., tip 14) relative to the target ROI across a series of imaging frames in the time sequence of images 35. In some embodiments, the velocity of the interventional instrument 12 (e.g., tip 14) relative to the target ROI may be determined by the speed of robot 16 traversing the path. In some embodiments, the processor 20 may predict the traversal time by detecting anatomical features of the portion of the anatomy (e.g., vessel tortuosity, bifurcations, vessel branching, vessel bends, small vessel diameter, calcification, lesions, etc. in the portion of the anatomy) and locations of the anatomical features in the anatomy in a series of imaging frames in the time sequence of images 35 in the prediction of such time. In some embodiments, the processor 20 may predict the traversal time based on characteristics of the interventional device (e.g., size, shape, material composition, flexibility, etc.) These embodiments may be combined to predict the traversal time by any combination of such distance, velocity, anatomical features, and device characteristics. In some embodiments, the processor calculates at least one statistical metric (e.g., mean, median, standard deviation) of the estimated time may be calculated for the predicted traversal time and a confidence level in the calculated metric(s) generated.

In some embodiments, for a given image of the time sequence of images, processor 20 may repeat the operations 106, 108, and 110 for several different ROIs. For example, in the time sequence of images 35, the processor 20 may determine a path from the location of the tip 14 to the location of the target ROI, and may identify a plurality of successive ROIs along the path. The processor 20 may then predict the traversal time to each of the successive ROI along the path, and may combine each traversal time to predict the traversal time for the entire path from the location of the tip 14 to the location of the target ROI. In this way, the processor 20 may estimates times to traverse various “way points” along the path taking into account the specific anatomical features (e.g., bends, etc.) of the corresponding segment of the path (e.g., between the “way point” and previous “waypoint”); specific characteristics of the interventional device (e.g., shape, etc.) while traversing the corresponding segment of the path; the specific distance along the corresponding segment of the path; the distance of the corresponding segment of the path; and/or etc.

With brief reference to FIG. 3, in some embodiments, the time estimation operation 108 can be performed by applying a machine-learning model (e.g., neural network (NN)) 36 stored in the non-transitory storage medium 26 of the electronic processing device 12. The machine-learning model 36 may have been trained on historical data, such as historical imaging data and historical patient data (for example, endovascular imaging data and endovascular patient data), to estimate the traversal time for the intervention device (e.g., tip 14 of the interventional device) to reach the target ROI. For example, the machine-learning model 36 may be trained to correlate relationships between features identified from the historical imaging data (e.g., distance from tip location to target ROI location, anatomical features between tip location to target ROI location, locations of the anatomical features between tip location to target ROI location, velocity of device indicated by historical imaging data, characteristics of the device, etc.) and/or historical patient data to corresponding transversal times for the device to travel from the tip location to the target ROI locations. For example, the machine-learning model may be trained to predict traversal times for respective segments of the path between the location of the interventional device and the target ROI based on the particular anatomical features (e.g., bends in a vessel) of the respective segments and/or the interventional device's capability of adapting to the anatomical features (e.g., flexibility to traverse a bend in a vessel). For example, the machine-learning may be trained to associate particular features of the anatomy with “slowdowns” in the navigation of the device resulting in an increased traversal time. As diagrammatically shown in FIG. 3, the trained machine-learning model 36 receives as input the current images 120 of the procedure including the interventional device and the target ROI 122, and predicts and outputs the traversal time for the interventional device 12 to reach the location of the target ROI 124. In some embodiment, the machine-learning model 36 may be trained with the time sequence of images 35.

At an operation 110, the processor 20 outputs the predicted traversal time, for example on the display device 24. In some examples, the processor 20 generates a visualization 38 of a path from the location of the interventional instrument 12 to the target ROI, and displays the visualization on the display device 24. The visualization 38 may include a visualization of the interventional instrument 12, the target ROI, an outline path from the tip 13 to the target ROI, one or more measurements (e.g., distance, velocity, etc.), anatomical features, device characteristics, and so forth.

With reference to FIG. 4, an illustrative example is shown of such a visualization on a graphical user interface (GUI) 28 for display on the display device 24. In this example, the image is a digital subtraction angiography (DSA) image obtained by acquiring images and subtracting to obtain the image highlighting the vasculature. In some embodiments, the images are acquired using intravascular contrast agent, and, in other embodiments, the images are acquired without using intravascular contrast agent. In FIG. 4, the operations 106, 108, and 110 are repeated for a plurality of successive ROIs along the path (labeled “Path” in FIG. 4) of the interventional instrument 12 from the identified current location of the tip 14 (“Current device position”) to a target ROI (“Target”, e.g., the clot C) which is the final ROI of the plurality of successive ROIs along the path. Each of the ROIs can be considered a “way point” along the path to the final target ROI. If the set of ROIs is denoted ROI₁, ROI₂ . . . , ROI_{N}, where ROI_{N} is the final target, then the traversal time from the current location of the tip to each ROIᵢ (where “i” is an index) can be denoted tᵢ(ROIᵢ). Then the traversal time from ROIᵢ to ROIᵢ₊₁ is given as tᵢ₊₁(ROIᵢ₊₁)−tᵢ(ROIᵢ). In this way, traversal times to traverse between various “way points” (ROIs) along the path can be computed or predicted, which can be converted to speeds or velocities as distance/time. As shown in FIG. 4, such speeds can be annotated to the visualization at the corresponding “way points” as traversal times (optionally represented as “speed” estimates). As seen in FIG. 4, a very sharp turn of the vasculature is estimated to requires a “Very slow speed” while other portions are labeled as “Average speed” or “Slightly slower speed”. Of course, these are illustrative annotations, and other formulations for the annotations are also contemplated. FIG. 4 also shows the displayed “Dist. to target: 23 mm” output by the optional operation 116 of FIG. 2, and the displayed “Time to target: 30 sec” output by the operation 110 of FIG. 2. As this image can be produced for a current image of the real-time imaging 102 of FIG. 2, as there indicated by feedback arrow 118 the visualization of FIG. 4 can be updated in real-time (e.g., every few seconds or faster) to provide the physician with an up-to-date estimate of the time-to-target as well as the traversal times.

Additionally, if a three dimensional (3D) image 112 of the vasculature is available, such as for example a pre-operative 3D computed tomography angiography (CTA) image or a pre-operative 3D magnetic resonance angiography (MRA) image, then in an operation 114 the tip-to-ROI distance can be estimated, and this information can also be displayed in an operation 116. In order to estimate the current device tip position relative to a pre-operative 3D image, a registration between the pre-operative 3D image and intra-operative image data may need to be performed. If the 3D image is acquired intra-operatively and the device tip is visible in the 3D image, then the registration step is not necessary. The estimated tip-to-ROI distance can be used to estimate the time for the tip 14 to reach the ROI in operation 108.

In general, the imaging 102 is real-time imaging performed over the course of the interventional procedure to provide imaging guidance for the physician guides the interventional instrument through the vasculature to position the tip 14 at the target ROI (e.g., clot C). Hence, the sequence of images spans a portion or all of the time over which the interventional instrument is inserted. The foregoing operations 104, 106, 108, and 110 (and optionally also 114 and 116) are thus optionally performed for successive images of this sequence of images to update the estimated time over the course of the insertion of the interventional instrument. This is indicated in FIG. 2 by a flowback arrow 118.

### EXAMPLE

The operations of the system 10 are described in more detail. The imaging device 30 can comprise an interventional X-ray imaging system consisting of an X-ray tube 32 adapted to generate X-rays and an X-ray detector 34 configured to acquire X-ray images 35. Examples of such systems are a fixed monoplane and biplane C-arm X-ray systems, mobile C-arm X-ray systems, etc. The X-ray imaging system can generate both regular fluoroscopy (X-ray) images as well as contrast enhanced fluoroscopy images containing the system 10.

In some embodiments, the location of the target region of interest and/or the location of the interventional device (e.g., tip) may be annotated by a user (e.g., via the touchscreen GUI 28). Alternatively, in other embodiments, the location of the target region of interest and/or the location of the interventional device (e.g., tip) may be automatically annotated by the processor 20. For example, the processor may automatically detect the target ROI (e.g., an aneurysm) in the time sequence of images 35 and annotate the detected target ROI. The annotation may be indicated using bounding box, centroid, binary segmentation, etc. The annotation may be generated using any of the methods (traditional computer vision-based or deep learning based) established in the art, including, thresholding, region growing, template matching, level sets, active contour modelling, neural networks (e.g., U-Nets), manual or semi-automatic methods, and so forth. The target location can also be provided as a label from a set of known labels (e.g., brain aneurysm) when the target location is not visible in the sequence of images. of the interventional device (e.g., tip 13) can be identified in the images 35 by, for example, segmentation, device tip detection, tracking devices (EM tracking), shape sensing processes, neural networks (e.g., U-Nets), and so forth.

Before use, the machine-learning model (e.g., NN 36) can be trained on training data. For example, the model may estimate the traversal time for the tip 14 to reach the target ROI based on features identified in previous images of the procedure, such as anatomical features, device characteristics, distance to the target ROI, velocity of device, etc. The estimated traversal time is then compared to a ground truth value. Parameters of the model may be adjusted based on a difference between estimated and ground truth value. These operations can be repeated until a stopping criterion is met.

To train the machine-learning model, retrospective navigation data from a relatively large (N>>100) patient population (covering various anatomies, abnormalities, age groups, genders, BMI, etc.) who have undergone any endovascular procedures may be received at the processing device 18. This retrospective navigation data may include for example, two-dimensional (2D) angiographic sequences of vasculature with identified device tip location and location of the target ROI. In some embodiments, both of which can be input into the model (e.g., NN) via an input channel consisting of: a binary mask of the device tip and/or target ROI; or a bounding box around the device tip or target ROI; or Gaussian heatmap centered at the device tip or target ROI, etc. Also, input into the model may be pre-op or intra-op three-dimensional (3D) images of the vasculature, if available, which provide more precise measurements of the path length and geometry of critical points, any available information that can be reliably collected and affect the device navigation (e.g., gender, age, blood pressure, weight, cardiac health, smoking history, family health history, treatment history, genomic data, etc.), or any available information about the device (e.g., speed) which could be acquired from, for example, a robotic navigation system 16, and so forth. In other examples, characteristics of the interventional device may be considered to train the model. For example, softer devices have a lower perforation risk at high velocity, but may present increased difficulty maneuvering into the vessel branches. In addition, a shape of the tip 14 can affect the time to target (for example, “pigtail” guidewires reduce the perforation risk of the system 10 at high velocities as compared to straight tips, and bent tips are easier to maneuver into vessel branches than straight tips).

The machine-learning model (e.g., NN 36) may then be trained using at least one 2D intraoperative image sequence, as well as any other optional information such as any 3D images of the vasculature or device speed provided from robotic device manipulation systems. In some embodiments, multiple 2D images inputted into the model must come from a continuous sequence (e.g., representing the same task or stage in the procedure), but can include, for example, regular fluoroscopy images and digital subtraction angiography (DSA) images from the same stage, images from different viewing angles (to provide additional 3D context), etc.

The NN 36 may be a convolutional neural network (CNN), a temporal convolutional network (TCN), a recurrent neural network (RNN), a transformer, or any other suitable artificial NN (ANN). An RNN-based implementation may use unidirectional or bidirectional long short-term memory (LSTM) architecture, etc. Temporal information when multiple images are input into the NN 36 can enable consistent and more accurate predictions across frames. The NN 36 may include several fully connected or convolutional layers with pooling, normalization, dropout, non-linear layers, etc. between them. Additional information may be incorporated by appending to flattened feature layers before applying non-linearities (e.g., sigmoid, ReLU, etc.).

The output of the machine-learning model is an prediction or estimate of the time to target, e.g., a prediction of the time it will take to navigate the interventional instrument 12 along a path from its current location, through the anatomy, to the target ROI. The machine-learning model may output a single value indicating the time to navigate the interventional instrument 12 along the path to the target ROI, or multiple outputs indicating the estimated time to navigate the interventional instrument 12 to several discrete points along the path to the target ROI. By training the machine-learning model to estimate the time to navigate to several discrete points towards the ROI, the model may be trained to associate features from the image with information relating to ease and time of navigation (what features are related with consistent slowdowns, what features might indicate scale, how path lengths are associated with time to target, etc.). In some embodiments, the output may be shown as a percentage when scale is not known (i.e., when only 2D information available) or in seconds (or other units) when scale is known (i.e., when 3D information is also available).

Errors are computed by comparing the outputs produced by the machine-learning model with ground truth values using some loss function (e.g., L1 or L2 loss, Huber loss, log cosh loss, etc.) and are used to perform stochastic gradient descent to optimize network weights. Ground truth values for time to target may be obtained by evaluating for each input image frame in the training data, the remaining time until the device tip 14 reaches the target ROI. This can be estimated using the location of the device tip 14, the location of target along with the framerate at which the image sequence 35 was captured, the features of the anatomy between the locations, characteristics of the device, etc.

Once trained, the machine-learning may be configured to receive at least one 2D angiographic image of the image sequence 35 containing the interventional instrument 12 at the current stage of the procedure with known device tip location. The location of the target ROI may be user-annotated or automatically annotated image images of the image sequence. Other data may also be received to estimate a distance, a velocity, anatomical features, device features, etc. associated with the navigation of the interventional instrument 12.

In some embodiments, the machine-learning model (trained with drop-out layers) may be run multiple times on the same input during inference to generate slightly different outputs (since dropout drops the outputs from a specified number of nodes at random). These different outputs may be used to compute the mean and variance in the time to target estimate. The variance may be used to indicate a level of confidence in the output (i.e., high variance indicates that network output is not consistent and, therefore, confidence is low, while low variance indicates consistent output and high confidence).

In some embodiments, the estimated time to navigate the device to several discrete points along the path to the ROI may be used to generate additional information by combining outputs across frames. For instance, by combining the time estimate across frames and computing how the estimate is changing, the estimated velocity of the device tip can be computed. Similarly, evaluating change in estimates across frames can indicate the location of potential slowdowns and the amount of slowdown.

The GUI 28 displays the 2D intra-op sequences of the vasculature and overlays the estimated time to target as a numerical value (if single value output) or as an annotated route to the target (if output consists of various discrete points along the path to the ROI). The route may be annotated by post-processed information (e.g., red where slowdowns may occur, green elsewhere; or red where low confidence, green where high confidence; etc.). The instantaneous velocity or the average velocity of the system 10 in each anatomical region may also be displayed.

In some embodiments, the machine-learning model may be trained to directly output areas of slowdown. For instance, in addition to the time to target estimates, the model may also generate an output the same size as in the input image with heatmaps indicating the potential locations of slowdown (e.g., high vessel tortuosity, bifurcations, small vessel diameter, etc.). This information can be derived directly from the ground truth time to target information and used during training.

In some embodiments, in the case that data from robotic navigation by the robot 16 is used for training, the estimated device velocity could be extracted from the robot 16 and used for calculating the other parameters such as time to target and amount of slowdown. The network could also be trained based on both manual and robotic navigation images, since robotic navigation data may be a bit different from manual navigation data. Training on data of both types is more likely to result in a more generalizable machine-learning model.

In some embodiments, if the images 35 include multiple C-arm views at the same time (e.g., data acquired from a biplane system), this data could be used by the machine-learning model to compute a more precise estimate of time to target or time to discrete points towards the target since this data would essentially provide more information about the path to the target and, hence, increase the confidence of the machine-learning model in its predictions. The multiple views may be inputted as separate input channels into the machine-learning model or as separate inputs into a Siamese twin type network architecture, where parallel convolutional layers process the multiple views separately in the early layers of the machine-learning model and merge the network weights in later layers to provide a combined output.

In some embodiments, if a path of the interventional instrument 12 is not defined, the output of the machine-learning model may be used to estimate a proper path from the current device location to the target ROI. That is, the estimated time to discrete points along the path to target can help inform how to get to the target ROI.

In some embodiments, instead of simply using the device tip 14, the machine-learning model uses information from the entire device visible in the images 35. This may provide additional information to the machine-learning model during training, for instance, what curvature of the device is related to fast and sudden movements of the device, etc.

In some embodiments, the machine-learning model is trained to directly output confidence values. The confidence here may be associated with training errors. This may allow the machine-learning model to learn features in images that typically result in higher errors. For instance, areas where vessels are foreshortened may typically generate higher errors during training due to the ambiguity presented by foreshortening and, therefore, may be associated with lower confidence values.

In some embodiments, the post-processed estimates may be customized to each user. For instance, the estimated amount of the slowdown could be refined based on the average device velocity as navigation proceeds (e.g., some users may navigate slower than the other users in general or due to the high risk of perforation for the patient).

In some embodiments, the output of the machine-learning model may be used to control the autonomous robot 16. For instance, identified areas of slowdown may be used to signal to the robot 16 to reduce translation speed since areas of slowdown may be areas with highly tortuous or narrow vessels that must be navigated carefully.

In some embodiments, when 3D information is also available, the distance to target can be computed, and this distance is displayed on the GUI 28.

In some embodiments, a baseline navigation time may be learned for different types of anatomy using data from experts. This baseline constitutes the time it should take to navigate through different parts of the anatomy to the target. If users elect to receive this information, their time to target may be compared against the baseline. This can help trainees learn which parts of the anatomy they are slower in and may help in isolating what techniques they should practice. This can also alert physicians to unexpected behavior if they have been navigating for some time without realizing that expected progress is not being made.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A system (10) for navigating an interventional instrument in an anatomy of a patient, the system comprising:
a processor and memory, the processor configured to:
receive one or more images (35) including a portion of an anatomy of a patient and an interventional instrument (12) disposed within the portion of the anatomy;
identify, from the one or more images, a location of the interventional instrument (12) within the portion of the anatomy and at least one anatomical feature of the portion of the anatomy;
identify a target region of interest (ROI) in the anatomy; and
predict a time to navigate the interventional instrument from the location of the interventional instrument, through the portion of the anatomy, to a location of the target ROI based on the at least one anatomical feature of the portion of the anatomy.

2. The system (10) of claim 1, wherein the at least one anatomical feature of the portion of the anatomy includes at least one of vessel tortuosity, a bifurcation, a vessel bend, small vessel diameter, and vessel branching in the portion of the anatomy.

3. The system (10) of claim 1, wherein the processor is further configured to predict the time based on at least one characteristic of the interventional instrument including at least one of size, shape, and material composition of the interventional instrument.

4. The system (10) of claims 1, wherein the processor (20) is further configured to:
determine a path to navigate the interventional device from the location of the interventional instrument, through the portion of the anatomy, to the location of the target ROI; and
identify a plurality of successive ROIs along the path and predict a time to navigate to each of the plurality of successive ROIS.

5. The system (10) of claim 4, wherein the processor (20) is further configured to:
generate a visualization (38) of the path to navigate the interventional instrument from the location of the interventional instrument, through the portion of the anatomy, to the location of the target ROI; and
display, on a display device (24), the generated visualization of the path.

6. The system (10) of claim 5, wherein the processor (20) is further configured to:
display an image of the one or more of images;
generate a graphical representation of the path superimposed on the displayed image; and
superimpose an annotation of the predicted time to navigate to each of the plurality of successive ROIs along the path on the graphical representation.

7. The system (10) of claim 1, wherein the processor (20) is further configured to:
calculate at least one statistical metric of the predicted time; and
generate at least one confidence level for the at least one statistical metric.

8. The system (10) of claim 1, wherein the processor is further configured to:
estimate, based on the one or more of images, a distance from the location of the interventional instrument, through the portion of the anatomy, to the location of the target ROI and predict the time to navigate based on the distance.

9. The system (10) of claim 1, wherein the processor is further configured to:
predict, based on a sequence of the one or more of images, a velocity of the interventional instrument from the location of the interventional instrument, through the portion of the anatomy, to the location of the target ROI and predict the time to navigate based on the velocity.

10. The system (10) of claim 1, wherein the processor is configured to apply a machine-learning model trained to predict the time to navigate from the location of the interventional instrument, through the portion of the anatomy, to the location of the target ROI based on the at least one anatomical feature of the portion of the anatomy.

11. The system (10) of claim 1, further comprising:
a robot (16) controlled by the processor (20) to move the interventional instrument (12) through the portion of anatomy of the patient at a velocity determined based on the predicted time.

12. The system (10) of claim 1, further comprising:
an imaging device (30) in communication with the processor, the imaging device configured to acquire the one or more of images.

13. The system (10) of claim 1, wherein a tip (14) of the interventional instrument (12) comprises a radiopaque material and the processor is further configured to identify the location of the interventional instrument in the one or more of images based on the radiopaque tip.

14. A method (100) for navigating an interventional instrument in an anatomy of a patient, the method comprising:
receiving one or more images (35) including a portion of an anatomy of a patient and an interventional instrument (12) disposed within the portion of the anatomy;
identifying, from the one or more images, a location of the interventional instrument (12) within the portion of the anatomy and at least one anatomical feature of the portion of the anatomy;
identifying a target ROI in the anatomy; and
predicting a time to navigate the interventional instrument from the location of the interventional instrument, through the portion of the anatomy, to a location of the target ROI based on the at least one anatomical feature of the portion of the anatomy.

15. The method (100) of claim 14, wherein the at least one anatomical feature of the portion of the anatomy includes at least one of vessel tortuosity, a bifurcation, a bend, small vessel diameter, and vessel branching in the portion of the anatomy.

16. The method (100) of claim 14, the time is predicted further based on at least one characteristic of the interventional instrument including at least one of size, shape, and material composition of the interventional instrument.

17. The method (100) of claims 14, further comprising:
determining a path to navigate the interventional device from the location of the interventional instrument, through the portion of the anatomy, to the location of the target ROI; and
identifying a plurality of successive ROIs along the path and predicting a time to navigate to each of the plurality of successive ROIs based on the at least one anatomical feature of the portion of the anatomy.

18. The method (100) of claim 14, further comprising:
estimating, based on the one or more of images, a distance from the location of the interventional instrument, through the portion of the anatomy, to the location of the target ROI and predicting the time to navigate based on the distance.

19. The method (100) of claim 14, further comprising:
predicting, based on a sequence of the one or more images, a velocity of the interventional instrument from the location of the interventional instrument, through the portion of the anatomy, to the location of the target ROI and predicting the time to navigate based on the velocity.

20. A non-transitory computer-readable storage medium having stored a computer program comprising instructions, which, when executed by a processor, cause the processor to:
receive one or more images (35) including a portion of an anatomy of a patient and an interventional instrument (12) disposed within the portion of the anatomy;
identify, from the one or more images, a location of the interventional instrument (12) within the portion of the anatomy and at least one anatomical feature of the portion of the anatomy;
identify a target ROI in the anatomy; and
predict a time to navigate the interventional instrument from the location of the interventional instrument, through the portion of the anatomy, to a location of the target ROI based on the at least one anatomical feature of the portion of the anatomy.
